(19) United States
(12) Patent Application Publication
(10) Pub. No.: US 2013/0089618 A1
(43) Pub. Date: Apr. 11, 2013

(54) Linseed Salve

(71) Applicant: Peggy Mary Miller, Missoula, MT (US)

(72) Inventor: Peggy Mary Miller, Missoula, MT (US)

(21) Appl. No.: 13/267,816

(22) Filed: Oct. 6, 2011

(51) Int. Cl. A61K 36/55; A61P 25/00; A61P 21/00; A61P 17/02; A61P 19/00

(52) U.S. Cl. 424/537

(57) ABSTRACT

Linseed Salve is a soothing, healing, all natural salve utilizing comfrey to heal, plantain to draw out the wound, mugwort as a nervine, calendula as a blood, moving diaphoretic and oils for their blood moving and drying abilities to clear old blood, and depleted, dried cells.
Comfrey is to speed healing of bones, tissues and muscles and control bleeding. It is a demulcent and hemostatic.
Plantain is an anti-inflammatory and calmative.
Calendula is an emmanagogue and diaphoretic which helps clear cellular debris from the wound.
Mugwort is a soothing nervine to address impact of shock and a diaphoretic for clearing heat.

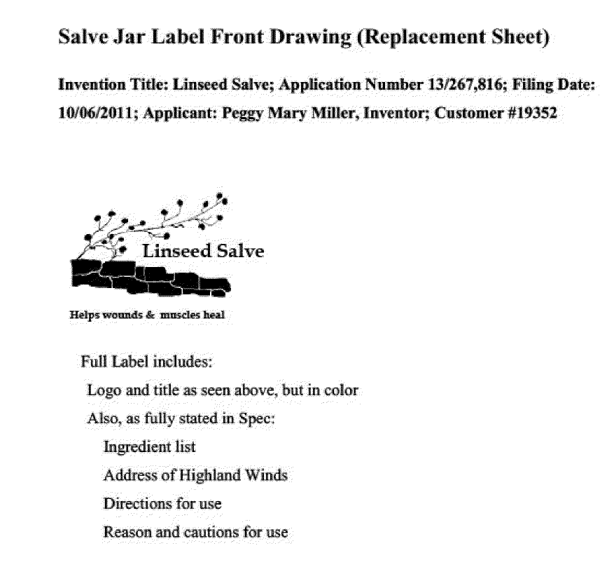

Safflower Oil is an emmanagogue and blood regulator and has drying tendencies which can help to dry the wounded area which promotes more rapid healing for its intended uses. Linseed Oil is a demulcent for inflammation and it helps dry the wound and offers directional penetration.

### THE SPEC

The below is a substitute specification since the previous spec did not include a title for the description of the drawing included in the application as pointed out to me in the Patent Office mailing of Feb. 13, 2012 and phone discussion Reference #1-205754365. I, Peggy Mary Miller, am attaching to this sheet the same information as included below without the underline markings to show where I included a title for the drawing description paragraph added to the specification and one correction. There is no new information in this overall application, simply additional clarifying explanation. It contains the same material as in the original application that I filed with you on tiling date: Oct. 6, 2011, and is in your records under application Ser. No. 13/267,816.

Linseed Salve:

1) Linseed Salve is a soothing, healing salve showing usefulness with pulled muscles, sprains, pinched nerve areas, phlegm deposits, bone bruises, cuts and abrasions. It is an ayurvedically made salve. Ingredient list:: Linseed Oil, Safflower Oil, Plantain, Calendula, Comfrey, Mugwort, Beeswax, Vit. E. Customers using the salve have called to say that it works well. Miss Miller has used it herself and on her animals, finding it speeds healing while it soothes. Due to its ability to speed healing, while helping to slow bleeding, and provide soothing relief it offers a unique usefulness in the health and wellness field.

### 2) BRIEF DESCRIPTION OF DRAWING

A drawing is included in the application which is a copy of the label design used on the jars that Linseed Salve is put into for distribution and sale. The label includes the name of the product, that it is a natural product that helps heal muscles, wounds, scrapes and tendons, an ingredient list as mentioned above, and directions for using the salve in an external manner, not for a prolonged time, and not by pregnant. An address of Highland Winds, Miss Miller's small business is on the label.

1: Linseed Salve is particularly useful for healing wounds, damaged bones, sprains, pulled muscles and pinched nerves in a natural manner.
This formula uses only natural elements for its ingredients and works to achieve healing by using the natural processes of the body which can digest and incorporate the various herbs and oils in this formula into its system without disrupting through use of synthetic elements, any natural flows of energy, blood, or water.

2: The particular mix of herbs in the formula, the quantity of each herb, as well as the types of oils used, help the formula to be one that, when rubbed on topically, naturally penetrates the skin, soothes, and begins the healing process quickly.
The skin has the ability to absorb ingredients through its outer layers, using its own natural moistures and the flow of these moistures and blood in the various layers, to incorporate the various herbs and oils in the mixture into the area that has been injured. The actual blend of herbs and oils chosen for this formula work together to help cells increase their rate of multiplying so that new cells can be located in the injured area, help the blood, diuretic and diaphoretic processes of the body rid the injured area of unwanted material from the injury including dried blood and damaged cells, sooth the nerves during the process thus causing increased relaxation in the area during the healing process. By doing so the herbs and oils work in conjunction so that healing is more rapid and thorough for the types of injuries mentioned above. It overall is a useful natural herbal product that would be good to have in every household.
The particular amount of each herb used, along with the amounts of oils in the formula, balance each other so that the herbs can complement each other as they work together. Mugwort is used in smaller amounts due to its main use as a nervine that soothes the area. Though Comfrey is a key herb, in that it speed the rate of healing, it works best with calendula and plantain helping to move the blood and cellular debris from the area while it helps the cells multiply at a faster rate. Safflower oil is a very good blood mover, while Linseed oil has a unique ability to dry the area of unwanted debris from the wound by working to draw out the area through its diaphoretic ability.
I did draw from a formula that was part of an herbal class I took at East West School of Planetary Herbalogy, which is how I am certified as an herbalist, but I used a volume rather than the weight measurement they called for in the class formula, which changed the amounts of the herbs used drastically, and I also used Linseed and Safflower Oils which were not part of the formula from the class as my choice of oils. Furthermore, I changed the proportions of herbs to oils. For that reason, I consider this invention solely mine.
Linseed Salve Formula:
Dry Herbs:
Calendula flowers—one part by volume
Plantain leaves—two parts by volume
Mugwort—½ part by volume
Comfrey leaves—one part by volume
(Example: if one part=8 oz=one cup dry measurement then this would be a total of 4 ½ cups of dry herbs.)
Oils:
These above dry herbs are added to following:
For every 6 oz. dry volume measurement of dry herbs use one pint of oil.
The oils are in proportions of 2 pints of Linseed Oil to One pint of Safflower Oil,
So above 4½ cups of dry herbs would be added to 4 pints of Linseed Oil and two pints of Safflower Oil.
Then: This is boiled for two hours covered.
Then beeswax is added to proper consistency which is about a ½ ounce of beeswax for every pint of oil after cooking it for two hours.
Finally, Vit E, approximately one teaspoon per every two pints of salve liquid is added.
